(19) United States
(12) Patent Application Publication
(10) Pub. No.: US 2026/0324663 A1
(43) Pub. Date: Oct. 22, 2026

(54) Method for processing plant products and corresponding processing unit

(71) Applicant: XEDA INTERNATIONAL, SAINT ANDIOL (FR)

(72) Inventors: Alberto SARDO, CHATEAURENARD (FR); Stéfano SARDO, CHATEAURENARD (FR)

(21) Appl. No.: 19/108,852

(22) PCT Filed: Sep. 8, 2023
(86) PCT No.: PCT/EP2023/074770

§ 371 Date: Mar. 5, 2025

(30) Foreign Application Priority Data
Sep. 8, 2022 (FR) FR2209006

(51) Int. Cl. A23B 7/157; A23B 7/005; A23B 7/10

(52) U.S. Cl. CPC A23B 7/157; A23B 7/0056; A23B 7/10

(57) ABSTRACT

The method is intended for the processing of plant products packaged in perforated containers. It comprises the following steps:
stacking containers (5) to form several stacks (7), each stack (7) having a height of between 1 and 2.5 m and a horizontal section of between 0.5 and 1.5 m²;
loading just one of the stacks (7) into a first processing booth (9);
showering the stack (7) loaded into the first processing booth (9);
unloading the stack (7) from the first processing booth (9);
loading just one of the stacks (7) into a second processing booth (15);
showering the stack (7) loaded into the second processing booth (15);
unloading the stack (7) from the second processing booth (15); the steps of loading and unloading the first processing booth (9) being performed during the step of showering the second processing booth (15), and the steps of loading and unloading the second processing booth (15) being performed during the step of showering the first processing booth (9).

The present invention relates to a method for processing plant products.

It is important that plant products such as fruit and vegetables do not lose their organoleptic qualities and retain an appealing appearance when placed on the market for quick consumption. Once harvested, plant products are usually stored for relatively long periods before being placed on the market.

Phenomena that can alter the appearance and taste of plant products include the proliferation of fungi and bacteria on their surface. This deterioration is even more rapid in the case of micro-wounds and cuts that appear on the skin during storage or handling of plant products. Another phenomenon that can damage plant products is scalding, which is manifested by a darkening of the skin of the plant products affected.

Various treatments for plant products are known, including thermotherapy, which involves applying a hot processing composition, such as water. Other treatments include the use of processing compositions containing fungicides such as eugenol.

FR 2909525 A 1 proposes a method and facility suitable for the thermotherapy of plant products. The plant products are packed in crates. They are treated in a large-volume showering booth. Means are provided in the booth for spraying the plant products contained in the boxes with hot water, possibly containing additives.

A high processing rate can be achieved with this method, due to the large volume of plant products that can be loaded inside the booth.

On the other hand, the heating power required to keep water hot is very high in a system of this type.

In this context, the aim of the invention is to propose a processing method and facility that makes it possible to reduce the heating power required, without compromising processing quality, while maintaining a high processing rate.

To this end, the invention according to a first aspect relates to a method for treating plant products packaged in perforated containers, the method comprising the following steps:
stacking the containers to form several stacks, each stack having a height of between 1 and 2.5 m and a horizontal section s of between 0.5 and 1.5 m²;
loading a single one of the stacks into a first processing booth comprising a belt of side walls, at least one of the side walls being deformable between a stack engagement configuration and a stack release configuration;
placing the or each deformable side wall of the first processing booth in the stack engagement configuration;
showering the stack loaded into the first processing booth with a processing composition having a temperature of between 30 °C and 60 °C, at a flow rate of between 160 and 300 m³/m²/h and for a period of between 10 s and 10 min;
unloading the stack from the first processing booth;
loading just one of the stacks into a second processing booth comprising a belt of side walls, at least one of the side walls being deformable between a stack engagement configuration and a stack release configuration;
placing the or each deformable side wall of the second processing booth in the stack engagement configuration;
showering the stack loaded into the second processing booth with a processing composition having a temperature of between 30 °C and 60 °C, at a flow rate of between 50 and 500 m³/m²/h and for a period of between 10 s and 10 min;
unloading the stack from the second processing booth;
the steps of loading and unloading the first processing booth being performed during the step of showering the second processing booth, and the steps of loading and unloading the second processing booth being performed during the step of showering the first processing booth.

Each processing booth has a limited volume, as it is designed to load a stack of low-volume containers. As a result, the flow rate of processing composition is significantly lower than in FR 2909525. The power required to heat this processing composition is therefore much lower.

Since the two processing booths are used alternately, with overlapping operations, the flow of liquid at each moment is moderate. However, the flow rate of liquid composition per m² and per hour is higher than in FR 2909525. The quality of the processing is improved.

The volume of plant products processed in each cycle in a processing booth is lower than in FR 2909525. However, because the two processing booths operate in overlapping cycles, processing capacity is only slightly affected.

The processing method may exhibit one or more of the following features, taken in isolation or in any combination that is technically possible:
each stack contains between one and six containers per level;
the first processing booth and the second processing booth are supplied from the same reservoir of processing composition;
the reservoir of processing composition is heated continuously;
the flow rate of processing composition at the showering step in the first processing booth and in the second processing booth is between 60 and 600 m³/h.

According to a second aspect, the invention relates to a unit for processing plant products packaged in perforated containers, the containers forming a plurality of stacks, each stack having a height of between 1 and 2.5 m and a horizontal section of between 0.5 and 1.5 m², the processing unit comprising:
a first processing booth sized to receive a single one of the stacks, the first processing booth comprising a belt of side walls, at least one of the side walls being deformable between a stack engagement configuration and a stack releasing configuration;
a second processing booth sized to receive a single one of the stacks, the second processing booth comprising a belt of side walls, at least one of the side walls being deformable between a stack engagement configuration and a stack releasing configuration;
a circuit for showering the stack loaded in the first processing booth with a processing composition;
a circuit for showering the stack loaded in the second processing booth with the processing composition;
means for heating the processing composition, and
a system for deforming the deformable walls of the first processing booth and a system for deforming the deformable walls of the second processing booth.

The processing unit may furthermore exhibit one or more of the following features, taken in isolation or in any combination that is technically possible:
the showering circuit of the first processing booth and the showering circuit of the second processing booth are supplied by the same reservoir of processing composition;
the heating means heat the processing composition contained in the reservoir;
the heating means have a power of between 500 and 1200 kW;
the flow rate of processing composition in the showering circuit of the first processing booth and in the showering circuit of the second processing booth is between 50 and 500 m³/m²/h.

Further features and advantages of the invention will be apparent from the detailed description given below, by way of indication and not in any way limiting, with reference to the appended figures, among which:

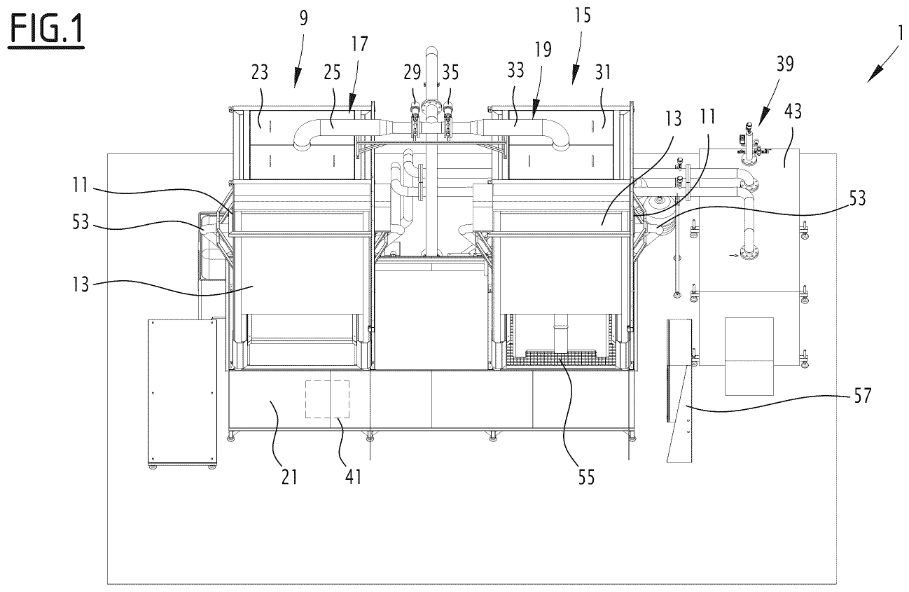

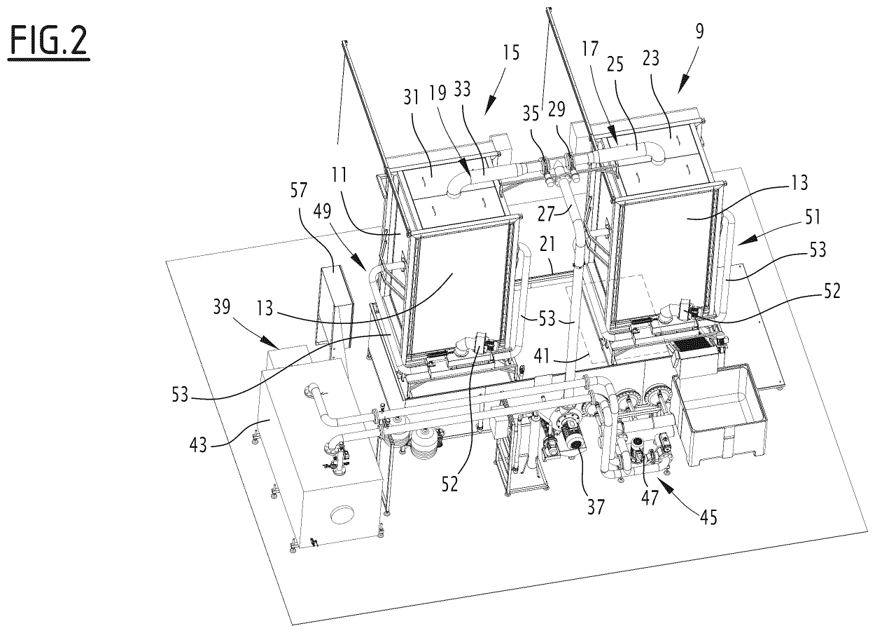

FIGS. 1 and 2 are perspective views of the processing facility of the invention, viewed from the front and rear respectively; and

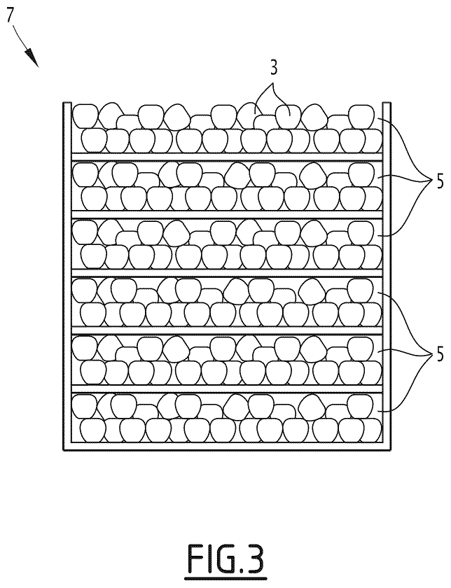

FIG. 3 is a schematic representation in elevation of a stack of containers loaded with plant products.

The processing unit 1 shown in FIGS. 1 and 2 is designed to process plant products 3 packed in perforated containers 5, the containers 5 forming several stacks 7 of the type shown in FIG. 3.

The plant products are fruit or vegetables.

The fruits may, for instance, be apples, pears or any other suitable type of fruit.

The vegetables may, for instance, be potatoes, or any other suitable type of vegetable.

The containers 5 are small, for example:
Type 1: length 1.2 m, width 1 m, height 0.65 m;
Type 2: length 1.2 m, width 1 m, height 0.35 m;
Type 3: length 0.45 m, width 0.30 m, height 0.30 m.

The containers are, for example, crates, pallet crates, or any other type of suitable container.

They are perforated in the sense that the processing composition sprayed onto the plant products packaged in the container can flow through the container. The bottom of the container in particular has holes (not shown) for the flow of the processing composition.

Each stack 7 is between 1 and 2.5 m high and has a horizontal section of between 0.5 and 1.5 m².

Each stack 7 is preferably between 1.5 and 2.5 m high and has a horizontal section of between 0.8 and 1.3 m².

Each stack 7 comprises between one and six containers 5 per level.

The stack 7 has between three and ten levels.

When the containers are larger, the stack 7 contains a single container 5 per level. It then has between three and five levels.

When the containers are smaller, the stack 7 typically comprises four or six containers 5 per level. It then has between six and ten levels.

For type 1 containers, the stack 7 comprises one container 5 per level and three levels.

For type 2 containers, the stack 7 comprises one container 5 per level and five levels. For type 1 containers, the stack 7 comprises six containers 5 per level and eight levels.

Each container 5 holds between 1 and 25 kg of plant products, preferably between and 20 kg.

The processing unit 1 comprises a first processing booth 9, sized to accommodate a stack 7 of the size indicated above, i.e. a stack 7 with a height of between 1 and 2.5 m and a horizontal section of between 0.5 and 1.5 m².

The first processing booth 9 internally defines a housing with a height and, perpendicular to the vertical direction, a cross-section corresponding substantially to the height and cross-section of a single stack 7.

The housing is thus adapted to accommodate a stack 7 with a height of between 1 and 2.5 m and a horizontal section of between 0.5 and 1.5 m².

The first booth 9 comprises a belt of side walls 11, 13, at least one of the side walls 11 being deformable between a stack 7 engagement configuration and a stack 7 release configuration.

The processing unit 1 further comprises a second processing booth 15, sized to accommodate a single stack 7.

The second booth 15 comprises a belt of side walls 11, 13, at least one of the side walls 11 being deformable between an engagement configuration and a release configuration of the stack 7.

The first processing booth 9 and the second processing booth 15 are preferably identical to each other.

Two of the side walls, referenced 11, are deformable between the engagement configuration and the release configuration. They face each other.

The other two side walls, 13, are rigid and typically made of sheet metal. They face each other.

One of the walls 13 is fixed and forms the rear of the processing booth.

The other wall 13 is a door allowing the inside of the processing booth to be accessed and the stack 7 to be loaded/unloaded. It forms the front of the processing booth. It is of any suitable type.

The walls 11 in the example embodiment are inflatable. Each of the two walls has a rigid outer surface and a flexible inner surface, for example made of polyester.

This wall 11 is inflated and moved into the engagement configuration by introducing a fluid such as compressed air between its inner and outer faces, which causes the inner face to press against the stack of crates 7 inside the housing.

The processing unit 1 further comprises a circuit 17 for showering the stack 7 loaded into the first processing booth 9 with a processing composition, and a circuit 19 for showering the stack 7 loaded into the second processing booth 15 with the same processing composition.

The showering circuits 17 and 19 are supplied by the same reservoir 21 of processing composition.

The reservoir 21 is a tank located under the two processing booths 9 and 15.

The showering circuit 17 comprises a spray bar 23 located in or on the booth 9, above the stack 7. It also comprises a pipe 25 fluidically connecting the ramp 23 to a manifold 27 for dispensing the processing composition.

A solenoid valve 29 is interposed in pipe 25, so as to authorise or prohibit the circulation of the processing composition from manifold 27 to distribution manifolds 23.

The showering circuit 19 for the stack 7 loaded into the second processing booth 15 is of the same type. It further comprises a spray bar 31 arranged in or on the booth, above the stack 7 of containers 5, and a pipe 33 connecting the spray bar 31 to the collector 27.

A solenoid valve 35, inserted into the pipe 33, authorises or prevents the flow of processing composition from the manifold 27 to the spray bar 31.

As can be seen in FIG. 2, the processing unit 1 comprises a member 37 for circulating the processing composition, the suction side of which is fluidically connected to the reservoir 21, and the discharge side of which is connected to the manifold 27.

The circulation member 37 is a pump, typically a centrifuge pump.

The unit 1 further comprises means 39 for heating the processing composition. In the example shown, the heating means 39 heat the processing composition contained in the reserve 21.

To achieve this, the heating means 39 typically comprise a heat exchanger 41 immersed in the reservoir 21. A heat transfer fluid circulates through the heat exchanger.

The heat exchanger 41 is shown schematically in FIG. 2. It is of any suitable type.

In this case, the heating means 39 comprise a boiler 43 configured to heat the heat transfer fluid, and a circuit 45 fluidically connecting the boiler 43 to the heat exchanger 41. The circuit 45 comprises a circulation device 47, such as a pump, which circulates the heat transfer fluid in a loop between the boiler 43 and the heat exchanger 41.

The boiler 43 is an electric boiler, a gas boiler, or any other type of boiler.

The unit 1 further comprises a system 49 for deforming the deformable walls 11 of the first processing booth 9 and a system 51 for deforming the deformable walls 11 of the second processing booth 15.

The two systems are identical.

Both systems are able to inflate the walls 11. They each comprise a blower 52 such as a fan, and ducts 53 connecting the outlet of the blower 52 to the volume bounded between the outer face and the inner face of the wall 11.

Each of the two processing booths 9, 15 is bounded at the bottom by a floor 55 immediately above the tank 21 (FIG. 1). This floor 55 is perforated to allow the processing composition to run from the internal volume of the booth into the reservoir 21. The stack 7 of containers is placed on the floor 55.

The heating means 39 have a heating power of between 500 and 1200 kW, preferably between 600 and 1000 kW, for example 800 kW.

The circulation member 37 is dimensioned to supply a flow of processing composition of between 60 m³ and 600 m³ per hour.

In each of the two processing booths 9, 15, the flow rate of processing composition in the showering step is between 50 m³ and 500 m³ per m² and per hour, preferably between and 300 m³ per m² and per hour.

This corresponds to a processing composition flow rate of between 60 m³ and 600 m³ per hour, distributed over the horizontal section of the booth, this section being approximately 1.2m², for example.

The processing unit 1 further comprises a controller 57, which controls, among other things, the shower circuits 17 and 19, the heating means 39, and the wall deformation systems 49 and 51.

The controller 57 particularly controls the circulation unit 37, the solenoid valves 29 and 35, the boiler 43, the circulation unit 47, and the blowers 51.

The controller 57 regulates the temperature of the processing composition within the range indicated above, and controls the flow rate of the processing composition so that it is within the range indicated above.

A method for treating plant products packed in perforated containers will now be described.

This method is designed to be implemented using the processing unit 1 described above. Conversely, the processing unit 1 is specially designed to implement the processing method.

The plant products packed in the containers are as described above.

The containers are perforated containers of the type described above.

The processing method comprises a step of stacking the containers 5 to form several stacks 7, each stack 7 having a height of between 1 and 2.5 m and a horizontal section of between 0.5 and 1.5 m².

Each stack 7 is preferably between 1.5 and 2.5 m high and has a horizontal section of between 0.8 and 1.3 m².

Each stack 7 comprises between one and six containers 5 per level.

The stack 7 has between three and ten levels.

The stacks are as described above.

The processing method further comprises the step of loading one of the stacks 7 into a first processing booth 9.

The first processing booth 9 comprises a belt of side walls 11, 13, at least one of the side walls 11 being deformable between a stack 7 engagement configuration and a stack 7 release configuration.

The first processing booth 9 is of the type described above.

The belt of side walls 11, 13 delimits the inside of a housing with a cross-section and height adapted to accommodate just one stack 7.

The method further comprises a step of placing the or each deformable side wall 11 of the first processing booth 9 in the stack engagement configuration, followed by a step of showering the stack 7 loaded into the first processing booth 9 with a processing composition.

The stack engagement configuration of the or each deformable side wall 11 is achieved by inflating the side wall 11 with a fluid such as air.

The air is forced into the internal volume of the side wall 11 as described above by the circulation member 51 of the system 49 for deforming the deformable walls 11.

In the engaged configuration, the inside of the wall 11 is pressed against the stack of containers 7. In addition, the stack 7 of containers rests against the fixed wall 13 at the rear and against the wall 13 acting as a loading door at the front.

In this way, the processing composition is forced to flow through the stack of perforated containers 7.

The stack 7 is showered by the showering circuit 17. The processing composition is sprayed by the spray bar 23 above the stack 7. It flows through the various containers 5 by gravity from the spray bar 23 to the grid 55.

At the end of the showering step, the method comprises a step of unloading the stack 7 from the first booth 9.

To do this, the loading door of the first booth 9 is opened, and an operator extracts the stack 7 from the booth.

It then loads a new stack 7 inside the first processing booth 9, and a new processing cycle inside the first processing booth 9 is started.

The method further comprises a step of loading one of the stacks 7 into a second processing booth 15.

The second processing booth 15 is as described above.

The method then comprises a step of placing the or each deformable wall 11 of the second processing booth 15 in the stack engagement configuration, and a step of showering the stack 7 loaded into the second processing booth 15 with the processing composition.

The steps of placing in the engagement and showering configuration are as described for the first processing booth 9.

At the end of the showering step, the method comprises a step of unloading the stack 7 from the second booth 15.

This unloading step is carried out as described above for the first booth 9.

The unloading step is followed by a step to load a new stack 7 into the second processing booth 15, and a new showering cycle inside the second processing booth 15 is then initiated.

Advantageously, the steps of loading and unloading the first processing booth 9 are carried out during the showering step in the second processing booth 15.

Conversely, the steps of loading and unloading the second processing booth 15 are carried out during the showering step in the first processing booth 9.

In this way, the loading and unloading of the first processing booth 9 are carried out in overlapping operations during the showering in the second processing booth 15.

Likewise, the loading and unloading of the second processing booth 15 are carried out in overlapping operations during the showering in the first processing booth 9.

The method in question therefore enables continuous processing to be carried out, without the processing unit having to be shut down completely to load or unload the stacks of containers. While one booth is stationary, the other is in operation.

The processing composition used for showering has a temperature of between 30 °C and 60 °C. Advantageously, it has a temperature of between 45 °C and 55 °C.

To do this, the reservoir 21 of processing composition is heated continuously. It is heated using heating means 39.

In each of the two processing booths, the flow rate of processing composition in the showering step is between 50 and 500 m³ per m² and per hour, preferably between 100 and 300 m³ per m² and per hour.

This corresponds to a processing composition flow rate of between 60 m³ and 600 m³ per hour, distributed over the horizontal section of the booth, this section being approximately 1.2 m², for example.

During the showering step in the first processing booth 9, the circulation member 37 is in operation. It sucks the processing composition from the reservoir 21 and pumps it into the showering circuit 17 of the first booth 9. The solenoid valve 29 is open, while the solenoid valve 35 serving the showering circuit 19 of the second booth 15 is closed.

Conversely, during the showering step in the second processing booth 15, the circulation device 37 is in operation, the solenoid valve 29 is closed and the solenoid valve 35 is open.

The showering step typically lasts between 10 seconds and 10 minutes, preferably between 1 and 5 minutes, and ideally between 2 and 3 minutes.

This processing composition is of any suitable type.

For example, the processing composition is water containing no particular active compound. Alternatively, the processing composition is water containing phosphonic acid (HPO₃H₂), also known as phosphorous acid H₃PO₃.

The method and the processing unit described above can have multiple variants.

Each processing booth can have a single deformable side wall, or more than two deformable side walls, for example three or four deformable side walls.

The processing unit could comprise more than two processing booths operating in overlapping cycles. The processing unit may therefore comprise three independent processing booths, or more than three independent processing booths. One or more of the processing booths are used for showering, while one or more other processing booths are being loaded/unloaded.

All the booths are arranged above the same reserve of processing composition, each booth being able to treat a single stack of containers.

A dedicated showering circuit can be set up for each booth, with its own circulation system.

The heating means may comprise an electrical heating element, such as a resistor, directly immersed in the reservoir of processing composition.

1. A method for treating plant products packaged in perforated containers, the method comprising the following steps:
stacking the containers to form several stacks, each stack having a height of between 1 and 2.5 m and a horizontal section of between 0.5 and 1.5 m²;
loading a single one of the stacks into a first processing booth comprising a belt of side walls, at least one of the side walls being deformable between a stack engagement configuration and a stack release configuration;
placing the or each deformable side wall of the first processing booth in the stack-engaging configuration;
showering the stack loaded into the first processing booth with a processing composition having a temperature of between 30 °C and 60 °C, at a flow rate of between 160 and 300 m³/m²/h and for a time of between 10 s and 10 min;
unloading the stack from the first processing booth;
loading a single one of the stacks into a second processing booth comprising a belt of side walls, at least one of the side walls being deformable between a stack engagement configuration and a stack release configuration;
placing the or each deformable side wall of the second processing booth in the stack-engaging configuration;
showering the stack loaded into the second processing booth with a processing composition having a temperature of between 30 °C and 60 °C, at a flow rate of between 50 and 500³/m²/h and for a time of between 10 s and 10 min;
unloading the stack from the second processing booth;
the steps of loading and unloading the first processing booth being performed during the step of showering the second processing booth, and the steps of loading and unloading the second processing booth being performed during the step of showering the first processing booth.

2. The method according to claim 1, wherein each stack comprises between one and six containers per level.

3. The method according to claim 1, wherein the first processing booth and the second processing booth are supplied from the same reservoir of processing composition.

4. The method according to claim 3, wherein the reservoir of processing composition is heated continuously.

5. The method according to claim 1, in which the flow rate of processing composition at the showering step in the first processing booth and in the second processing booth is between 60 and 600 m³/h.

6. A unit for processing plant products packaged in perforated containers, the containers forming several stacks, each stack having a height of between 1 and 2.5 m and a horizontal section of between 0.5 and 1.5 m², the processing unit comprising:
a first processing booth of a size adapted to receive a single one of the stacks, the first processing booth comprising a belt of side walls, at least one of the side walls being deformable between a stack engagement configuration and a stack release configuration;
a second processing booth of a size adapted to receive a single one of the stacks, the second processing booth comprising a belt of side walls, at least one of the side walls being deformable between a stack engagement configuration and a stack release configuration;
a circuit for showering the stack loaded into the first processing booth with a processing composition;
a circuit for showering the stack loaded into the second processing booth with the processing composition;
means for heating the processing composition, and
a system for deforming the deformable walls of the first processing booth and a system for deforming the deformable walls of the second processing booth.

7. The processing unit according to claim 6, wherein the showering circuit of the first processing booth and the showering circuit of the second processing booth (15) are supplied by the same reservoir of processing composition.

8. The processing unit according to claim 7, wherein the heating means heat the processing composition contained in the reservoir.

9. The processing unit according to claim 6, wherein the heating means have a power of between 500 and 1200 kW.

10. The processing unit according to claim 6, in which the flow rate of processing composition in the showering circuit of the first processing booth and in the showering circuit of the second processing booth is between 50 and 500 m³/m²/h.
